(19) United States
(12) Patent Application Publication
(10) Pub. No.: US 2010/0076409 A1
(43) Pub. Date: Mar. 25, 2010

(54) CATHETER CONNECTORS, CONNECTOR ASSEMBLIES AND IMPLANTABLE INFUSION DEVICES INCLUDING THE SAME

(76) Inventor: Lawrence Scott Ring, Valencia, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/234,416

(22) Filed: Sep. 19, 2008

(51) Int. Cl. A61M 25/18

(52) U.S. Cl. 604/533

(57) ABSTRACT

Catheter connectors and connector assemblies that are configured to reduce the likelihood of ESC cracks.

### BACKGROUND

1. Field of Inventions The present inventions relate generally to connectors that may be used to connect a catheter to a catheter or other device.

2. Description of the Related Art

Implantable infusion devices have been used to provide patients with a medication or other substance (collectively “infusible substance”) and frequently include an implantable pump and a catheter. A reservoir stores the infusible substance within the pump and, in some instances, implantable pumps are provided with a fill port that allows the reservoir to be transcutaneously filled (and/or re-filled) with a hypodermic needle. The reservoir is coupled to a fluid transfer device within the pump which is, in turn, connected to a catheter connector that functions as an outlet port. The catheter, which has at least one outlet, may be connected to a catheter connector on the pump. As such, the infusible substance may be transferred from the reservoir to the target body region by way of the fluid transfer device and catheter.

In some implantable infusion devices, the catheter that is located within target body region is not directly connected to the implantable pump. In the context of infusible substance delivery into the subarachnoid space around the spinal cord or brain, the catheters tend to be relatively long, thin and soft. Some physicians are of the opinion that, were the subarachnoid catheter to extend from the target location to the implantable pump, the portion of the catheter that extends from the spine to the implantable pump would be susceptible to kinks and damage from sutures. Accordingly, one common practice is to connect the subarachnoid catheter to a relatively thick proximal catheter that, in turn, is connected to the implantable pump.

Many of the catheter connectors that are used to connect a catheter to an implantable pump, or to connect one catheter to another, include a support tube that is larger in diameter than the unstretched inner diameter of the catheter for which it is intended. The difference in diameter results in the catheter exerting a radial force on the support tube after the catheter is pushed onto the support tube that, in turn, results in a friction force that tends to hold the catheter on the support tube. A barb, with a sharp edge that extends around the entire circumference of the support tube, may also form part of the connector. The sharp edge, which is followed by a region of reduced diameter, will engage the catheter as the catheter is pulled in the removal direction. The catheter will stretch and the stretching, but for the presence of the support tube, would cause the catheter to “neck down,” i.e. would cause the inner and outer diameters of the catheter shrink. As such, the radial force exerted by the catheter increases, as will the friction force, in response to the catheter being pulled in the removal direction.

The present inventor has determined that conventional barbed connectors are susceptible to improvement. For example, the present inventor has determined that a sharp edge that extends all the way around the barb and support tube creates a stress riser which extends all the way around the catheter at the sharp edge. The present inventor has further determined that a stress riser which extends all the way around the catheter at the sharp edge makes the entire circumference of the catheter at the sharp edge unnecessarily susceptible to environmental stress corrosion (ESC) cracks.

Some barbed connectors are used in connector assemblies which also include a strain relief element that is positioned over the portion of the catheter associated with the barbed connector. The present inventor has determined that conventional connector assemblies are susceptible to improvement. For example, the present inventor has determined that conventional connector assemblies allow cells to adhere to the outside of the catheter at the high stress area associated with the sharp edge of the barb. The cells excrete an acidic material that contributes to ESC cracks at the sharp edge.

### SUMMARY

A connector in accordance with one implementation of a present invention includes a support tube and a barb with one or more sharp edges that do not extend all the way around the support tube. There are a variety of advantages associated with such a connector. For example, the present configuration provides one or more sharp-edged regions that initiate the “neck down” when the associated catheter is pulled in the removal direction, and also provides one or more regions that are longitudinally aligned with, and impart less stress than, the sharp-edged regions. The catheter regions under less stress are less likely to suffer from ESC cracks and, accordingly, will preserve the integrity of the connection should cracks form in the regions associated with the sharp edges.

A connector in accordance with one implementation of a present invention includes a support tube and a barb with first and second barb members. The first barb member includes an apex without a sharp edge and the second barb member include at least one sharp edge that is longitudinally spaced from the apex of the first barb member. So configured, the region of the associated catheter that may be susceptible to ESC cracking will not effect the region that is forming the seal.

A connector assembly in accordance with one implementation of a present invention includes a connector, with a support tube and a barb, and a strain relief element. The assembly is configured such that a seal is formed between the strain relief element and the catheter with the inner surface of the strain relief element and the outer surface of at least a portion of the catheter section over the barb. Such a seal prevents cells from adhering to the portion of the catheter that is aligned with the barb and subject to ESC cracking.

A method in accordance with one implementation of a present invention includes creating a seal between a strain relief element and a catheter with the inner surface of the strain relief element and the outer surface of at least a portion of the catheter section that is over a barb. The creation of such a seal prevents cells from adhering to the portion of the catheter that is aligned with the barb and subject to ESC cracking.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a representation of an implantable infusion device having a catheter that is located within the subarachnoid space in accordance with one embodiment of a present invention.

FIG. 2 is a section view of a catheter that is located within the subarachnoid space.

FIG. 3 is a section view of a connector assembly in accordance with one embodiment of a present invention.

FIG. 4 is a perspective view of a connector in accordance with one embodiment of a present invention.

FIG. 5 is a section view taken along line 5-5 in FIG. 4.

FIG. 6 is another section view taken along line 5-5 in FIG. 4.

FIG. 7 is an enlarged side, partial section view of a catheter and a portion of the connector illustrated in FIG. 4.

FIG. 8 is a section view taken along line 8-8 in FIG. 7.

FIG. 9 is a section view taken along line 9-9 in FIG. 7.

FIG. 9A is a side view of a portion of a connector in accordance with one embodiment of a present invention.

FIG. 9B is a plan view of the connector illustrated in FIG. 9A.

FIG. 9C is a side view of a portion of a connector in accordance with one embodiment of a present invention.

FIG. 9D is a plan view of the connector illustrated in FIG. 9C.

FIG. 9E is a side view of a portion of a connector in accordance with one embodiment of a present invention.

FIG. 9F is a plan view of the connector illustrated in FIG. 9E.

FIG. 9G is a side view of a portion of a connector in accordance with one embodiment of a present invention.

FIG. 9H is a side view of a portion of a connector in accordance with one embodiment of a present invention.

FIG. 9I is a plan view of the connector illustrated in FIG. 9H.

FIG. 10 is a section view of the connector assembly illustrated in FIG. 3 in a disconnected state.

FIG. 11 is a perspective view of an exemplary catheter.

FIG. 12 is a section view taken along line 12-12 in FIG. 11.

FIG. 13 is another section view taken along line 12-12 in FIG. 11.

FIG. 14 is a plan view of an implantable infusion device in accordance with one embodiment of a present invention.

FIG. 15 is a section view of a connector assembly in accordance with one embodiment of a present invention in a disconnected state.

FIG. 16 is a section view of the connector assembly illustrated in FIG. 15 in a connected state.

FIG. 17 is an enlarged view of a portion of FIG. 16.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to use with the exemplary implantable infusion device described herein and, instead, are applicable to other implantable or otherwise ambulatory infusion devices that currently exist or are yet to be developed.

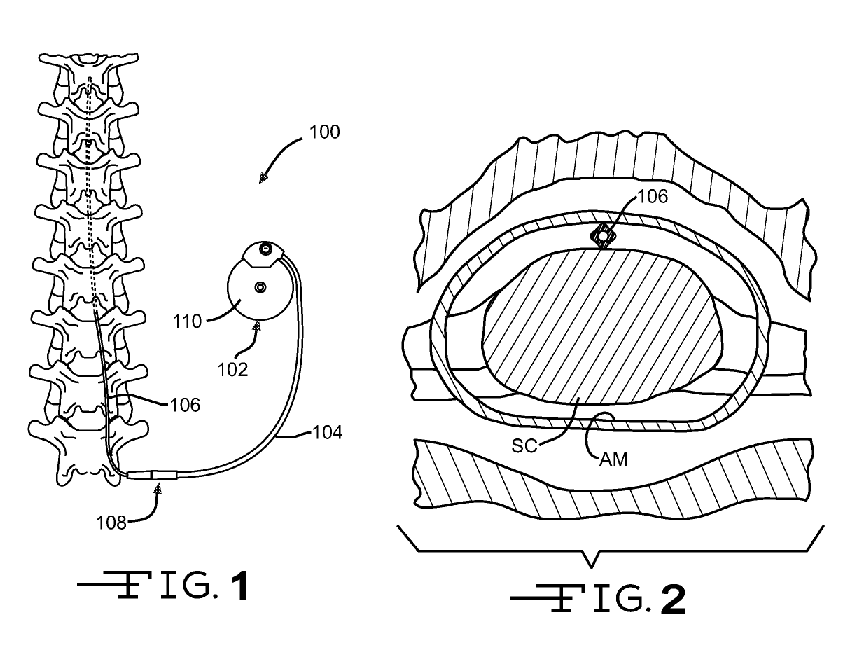

One example of an implantable infusion device in accordance with a present invention is generally represented by reference numeral 100 in FIG. 1. The implantable infusion device 100 includes an implantable pump 102, a proximal catheter 104 that is connected to the pump, a subarachnoid catheter 106, and a connector assembly 108. The implantable pump 102 includes a housing 110. An infusible substance reservoir, a fluid transfer device, control electronics and various other devices are carried within the housing 110. Although the present inventions are not limited to any particular type of implantable pump, exemplary pumps are described in U.S. Patent Pub. Nos. 2005/0273083 and 2006/0270983, which are incorporated herein by reference. The connector assembly 108 may be used to connect the proximal catheter 104 to the subarachnoid catheter 106 before or after the subarachnoid catheter has been positioned within the patient's body. For example, in those instances where a stylet is used to push the distal portion of the subarachnoid catheter 106 to the target location, the subarachnoid catheter will be connected to the proximal catheter 104 after the stylet has been removed. The infusible substance may then be delivered to, for example, the portion of the subarachnoid space along the spine between the spinal cord SC and the arachnoid mater AM, as is illustrated in FIG. 2.

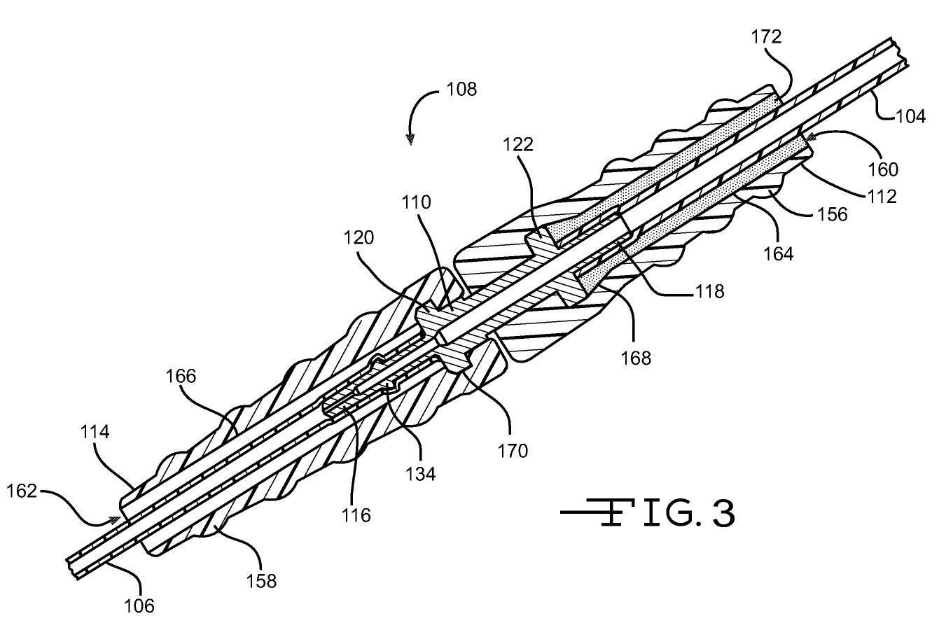

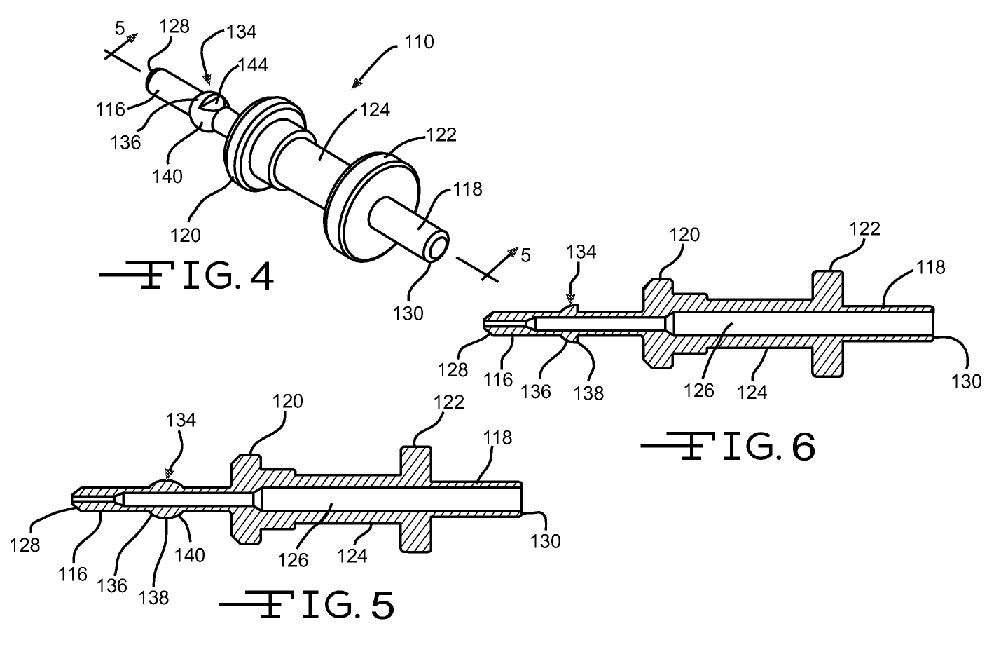

As illustrated for example in FIGS. 3-6, the exemplary connector assembly 108 includes a connector 110, a first strain relief element 112 and a second strain relief element 114. The connector 110 has first and second support tubes 116 and 118, first and second flanges 120 and 122, and a central portion 124. A lumen 126 extends from the free end 128 of the first support tube 116 to the free end 130 of the second support tube 118. Although connectors in accordance with the present inventions are not limited to any particular shape, the first and second support tubes 116 and 118 and central portion 124 are generally cylindrical in shape, while the first and second flanges 120 and 122 are disk-shaped. The lumen 126 is also cylindrical in shape, but for the frusto-conical regions associated with changes in lumen diameter. The diameter of the lumen 126 may vary as shown, may vary in other ways, or may be uniform from one end of the connector 110 to the other. A barb 134 is located between the flange 120 and the free end 128. The barb 134 may be an integral part of the support tube 116 (as shown) or a separate structure that is mounted on the support tube.

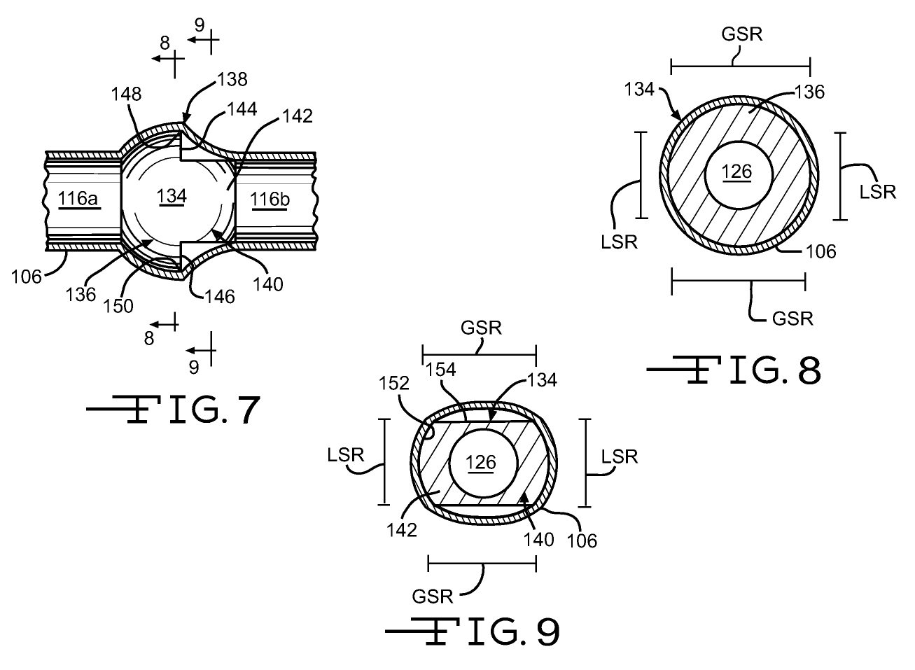

The overall shape of the exterior of the exemplary barb 134 is generally that of a sphere and, more specifically, that of a zone of a sphere which begins and ends where the outer surface of the barb intersects the outer surface of the support tube 116. To that end, and as illustrated in FIGS. 4-9, the exemplary barb 134 includes a first barb section 136, which gradually increases in size from outer surface 116a of the support tube 116 to an apex 138, and a second barb section 140. Referring more specifically to FIGS. 7-9, the second barb section 140 includes a mid-portion 142, which gradually decreases in size from the apex 138 to the outer surface 116b, and a pair of indentations 144 and 146 that extend to the apex. The second barb section mid-portion 142 creates a gradual transition from the apex 138 to the outer surface 116b. The indentations 144 and 146, on the other hand, result in abrupt transitions and a pair of sharp edges 148 and 150 at the apex 138 that are separated from one another by the mid-portion 142. So configured, the perimeter of the barb 134 that is aligned with the sharp edges 148 and 150 and is perpendicular to the longitudinal axis of the support tube 116 has regions with sharp edges and regions without sharp edges.

The exemplary first and second barb sections 136 and 140 define different shapes in cross-sections taken in planes perpendicular to the longitudinal axis of the support tube 116. Cross-sections taken in planes perpendicular to the longitudinal axis and within the first barb section 136 have a circular perimeter (FIG. 8). Cross-sections taken in planes perpendicular to the longitudinal axis and within the second barb section 140 have a non-circular perimeter and, in particular, have a pair of curved perimeter portions 152 separated by a pair of flat perimeter portions 154 (FIG. 8). So configured, the first barb section 136 performs the function of stretching the associated portion of the catheter 106 into a cross-sectional shape (i.e. a circle in the illustrated embodiment) that has a perimeter, while the second barb section 140 performs the function of stretching the associated portion of the catheter into a different cross-sectional shape (i.e. two partial circles combined with two arcs in the illustrated embodiment) that also has a perimeter. The perimeters associated with each of the barb sections 136 and 140, which are measured in planes perpendicular to the longitudinal axis, are larger on the first barb section side of the apex 138 than they are on the second barb section side of the apex, when measured at points along the longitudinal axis that are equidistant from the apex.

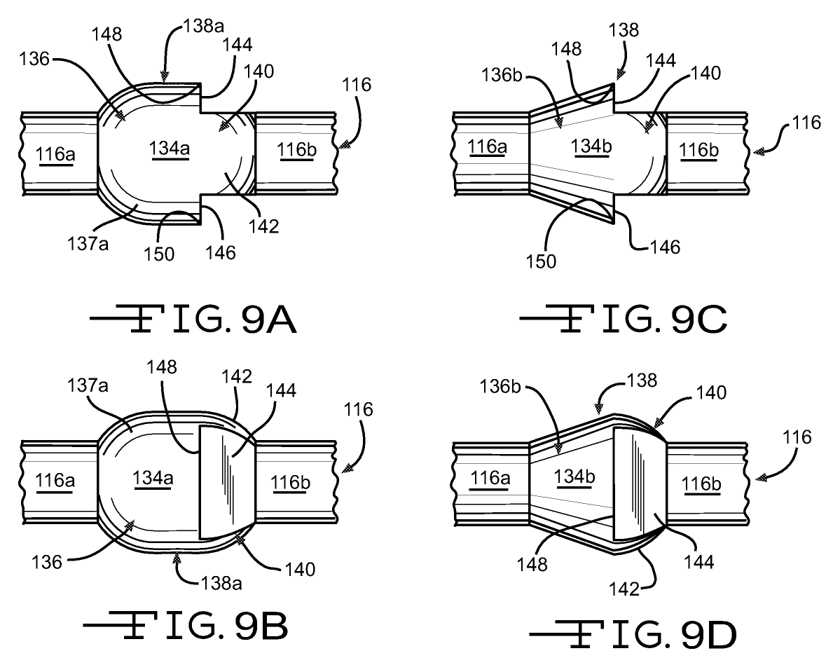

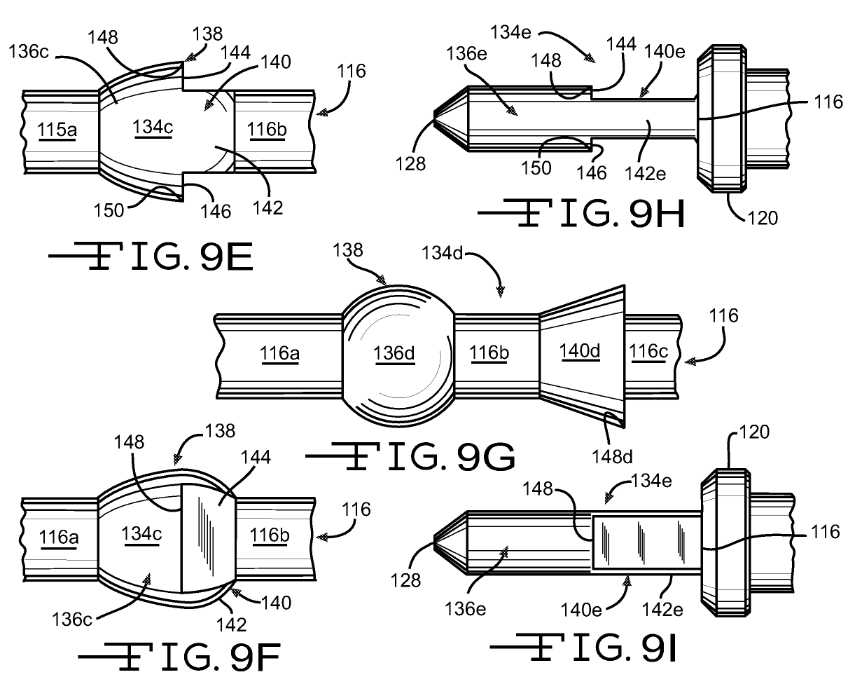

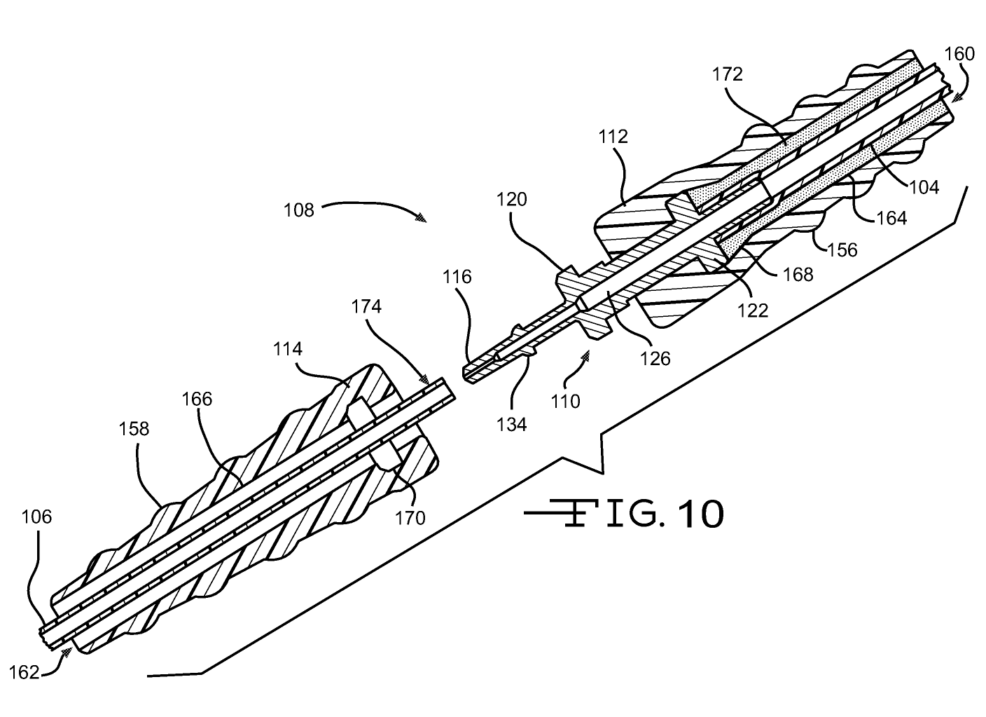

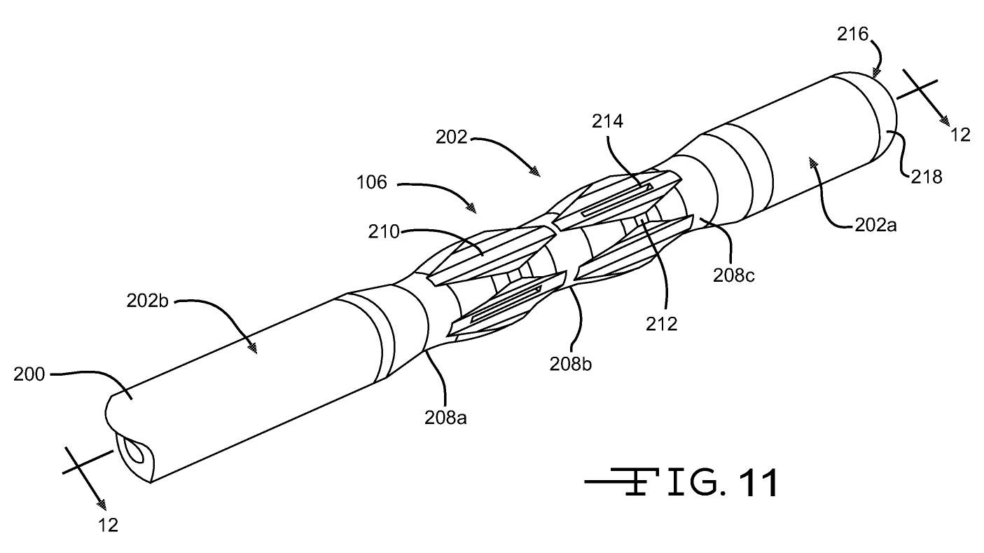

The exemplary barb 134 also performs the function of creating different levels of stress at longitudinally aligned regions of the catheter 106. As used herein, “longitudinally aligned” regions are regions that are spaced circumferentially (or are spaced about a non-circular perimeter) and also extend longitudinally along the same portion of the longitudinal axis of the catheter. In the illustrated implementation, the regions of the catheter 106 that extend over both the first barb section 136 and the mid-portion 142 of the second barb section 140 are not subject to the stress concentrations associated with the indentations 144 and 146 and the sharp edges 148 and 150. The regions of the catheter 106 that extend over both the first barb section 136 and the mid-portion 142 of the second barb section 140 are, therefore, under less stress than the regions of the catheter that extend over both the first barb section 136 and the indentations 144 and 146 of the second barb section 140. The regions of the catheter 106 under less stress, which are labeled LSR in FIGS. 8 and 9, will preserve the integrity of the connection between the catheter and the connector 110 when there are failures at regions under greater stress, which are labeled GSR in FIGS. 8 and 9. As the GSR regions deteriorate with age and stress, the LSR regions retain their strength and remain viable. These strips of LSR region material prevent the tubing from falling off of the connector tube.

Other exemplary barbs which perform the functions described above and below in the context of barb 134 are generally represented by reference numerals 134a-134e in FIGS. 9A-9I. Many aspects of barb 134 are incorporated into barbs 134a-134e and similar aspects are represented by similar reference numerals. Barbs 134a-134e may be incorporated into connectors (e.g. connector 110) and/or connector assemblies (e.g. connector assembly 108) in manner described above and below in the context of barb 134.

The overall shape of the exemplary barb 134a illustrated in FIGS. 9A and 9B is generally that of two hemispheres separated by a cylinder. More specifically, the shape includes a first zone of a sphere, which begins where the outer surface of the barb intersects the outer surface of the support tube 116 and ends at a cylinder, the cylinder itself, and a second zone of a sphere, which begins at the cylinder and ends where the outer surface of the barb intersects the outer surface of the support tube. To that end, the exemplary barb 134a includes a first barb section 136, which gradually increases in size from outer surface 116a of the support tube 116 to a cylindrical section 137a (which defines the apex 138a of the barb), and a second barb section 140. The second barb section 140 includes a mid-portion 142, which gradually decreases in size from the apex 138a to the outer surface 116b, and a pair of indentations 144 and 146 that extend to the cylindrical section 137a. The second barb section mid-portion 142 creates a gradual transition from the cylindrical section 137a to the outer surface 116b. The indentations 144 and 146, on the other hand, result in abrupt transitions and a pair of sharp edges 148 and 150 at the cylindrical section 137a that are separated from one another by the mid-portion 142. So configured, the perimeter of the barb 134a that is aligned with the sharp edges 148 and 150 and is perpendicular to the longitudinal axis of the support tube 116 has regions with sharp edges and regions without sharp edges.

The overall shape of the exemplary barb 134b illustrated in FIGS. 9C and 9D is generally that of a cone combined with a hemisphere. More specifically, the shape includes a frusto-conical portion which begins where the outer surface of the barb intersects the outer surface of the support tube 116, and a zone of a sphere which begins at the frusto-conical portion and ends where the outer surface of the barb intersects the outer surface of the support tube. To that end, the exemplary barb 134b includes a first barb section 136b, which is frusto-conical in shape and gradually increases in size from outer surface 116a of the support tube 116 to the apex 138, and a second barb section 140. The second barb section 140 includes a mid-portion 142, which gradually decreases in size from the apex 138 to the outer surface 116b, and a pair of indentations 144 and 146 that extend to the apex 138. The second barb section mid-portion 142 creates a gradual transition from the apex 138 to the outer surface 116b. The indentations 144 and 146, on the other hand, result in abrupt transitions and a pair of sharp edges 148 and 150 at the apex 138 that are separated from one another by the mid-portion 142. So configured, the perimeter of the barb 134b that is aligned with the sharp edges 148 and 150 and is perpendicular to the longitudinal axis of the support tube 116 has regions with sharp edges and regions without sharp edges.

The overall shape of the exemplary barb 134c illustrated in FIGS. 9E and 9F is generally that of a spheroid combined with a hemisphere. More specifically, the shape includes a zone of a spheroid which begins where the outer surface of the barb intersects the outer surface of the support tube 116, and a zone of a sphere which begins at the zone of a spheroid and ends where the outer surface of the barb intersects the outer surface of the support tube. To that end, the exemplary barb 134c includes a first barb section 136c, which has a truncated spheroid shape and gradually increases in size from outer surface 116a of the support tube 116 to the apex 138, and a second barb section 140. The second barb section 140 includes a mid-portion 142, which gradually decreases in size from the apex 138 to the outer surface 116b, and a pair of indentations 144 and 146 that extend to the apex 138. The second barb section mid-portion 142 creates a gradual transition from the apex 138 to the outer surface 116b. The indentations 144 and 146, on the other hand, result in abrupt transitions and a pair of sharp edges 148 and 150 at the apex 138 that are separated from one another by the mid-portion 142. So configured, the perimeter of the barb 134c that is aligned with the sharp edges 148 and 150 and is perpendicular to the longitudinal axis of the support tube 116 has regions with sharp edges and regions without sharp edges.

Turning to FIG. 9G, the exemplary barb 134d includes two barb members that may be contiguous or non-contiguous (as shown) and arranged such the region of the catheter that is aligned with the barb and is under less stress will be longitudinally spaced from the region of the catheter that is aligned with the barb and is under greater stress. As such, the region of the catheter that may be susceptible to ESC cracking will not effect the region that is forming the seal. The shape of the first barb member 136d is generally that of a sphere and, more specifically, that of a zone of a sphere which begins and ends where the outer surface of the barb intersects the outer surface of the support tube 116. The first barb member 136d does not include indentations and/or sharp edges. Instead, the first barb member 136d gradually increases in size from outer surface 116a of the support tube 116 to an apex 138, and gradually decreases in size from the apex 138 to the outer surface 116b. The first barb member 136d is not limited to spherical shapes and may alternatively be shaped, for example, like the barbs illustrated in FIGS. 9A-9F (albeit without the indentations). The second barb member 140d gradually increases in size and defines a sharp edge 148d that extends completely around the barb member. The second barb member 140d is not limited to the illustrated frusto-conical shape and may also be shaped, for example, like the barbs illustrated in FIGS. 7 and 9A-9F (albeit with a sharp edge that extends all the way around the barb). It should also be noted that the first and second barb members 136d and 140d may be the same size (as shown) or may be differently sized, as applications so require.

Another exemplary barb is generally represented by reference numeral 134e in FIGS. 9H and 9I. The barb 134e extends from one end of the underlying support tube 116 to the other, i.e. from the flange 120 to the free end 128. The barb 134e also includes a first barb section 136e, which has a constant cross-sectional size and shape (e.g. cylindrical) over its length, and a second barb section 140e. The second barb section 140e includes a mid-portion 142e, which has a constant cross-sectional size and shape, and a pair of indentations 144 and 146. The indentations 144 and 146 result in abrupt transitions and a pair of sharp edges 148 and 150 at the end of the first barb section and are separated from one another by the mid-portion 142e. So configured, the perimeter of the barb 134e that is aligned with the sharp edges 148 and 150 and is perpendicular to the longitudinal axis of the support tube 116 has regions with sharp edges and regions without sharp edges.

Referring now to FIGS. 3 and 10, the first and second strain relief elements 112 and 114 are flexible structures formed from materials such as silicone rubber, polyurethane, and other soft polymeric materials, and may include protrusions 156 and 158 or other structures that enhance the physicians ability to grip the strain relief elements during a surgical procedure. The first and second strain relief elements 112 and 114 also include internal lumens 160 and 162. The internal lumens 160 and 162 have relatively narrow regions 164 and 166 for the catheters 104 and 106 and relatively wide regions 168 and 170 for the connector flanges 122 and 120. The relatively wide regions 168 and 170 and the connector flanges 122 and 120 together removably secure the first and second strain relief elements 112 and 114 to the connector 110.

In some instances, it may be convenient to permanently connect one of the catheters to a portion of the exemplary connector assembly 108. The abdominal implantation of an infusion device, such as the exemplary infusion device 100 that includes a subarachnoid catheter 106 (FIG. 1), is one example of an instance where a permanent connection is convenient. Here, the subarachnoid catheter 106 may be disconnected from the connector 110 so that a stylet may be temporarily positioned within the catheter lumen and used to push the distal portion of the catheter to the target location. There is, on the other hand, often no reason to connect/disconnect the proximal catheter 104 and strain relief element 112 from the connector 110. As such, adhesive 172 may be used to permanently connect the proximal catheter 104 to the connector 110 and strain relief element 112 prior to the surgical procedure.

While the catheter 106 is disconnected from the connector 110 in the manner illustrated in FIG. 10, the strain relief element 114 may be positioned distal of the proximal portion 174 of the catheter 106 so that the catheter may be grasped by the clinician and pushed over the support tube 116 and barb 134 to connect the catheter to the connector 110. The strain relief element 114 may then be pushed over the connector flange 120 to secure the strain relief element to the connector.

Turning to FIGS. 11-13, and although the present connectors and connector assemblies are not limited to use with any particular type of catheter, one example of a catheter that may be used in combination with the connector assembly 108 is the exemplary subarachnoid catheter 106. The subarachnoid catheter 106 includes a catheter body 200 with a distal portion 202 and a central lumen 204 that extends from the proximal end of the catheter (i.e. the end adjacent to the connector assembly 108 in FIG. 1) to the distal end 206 of the catheter. The catheter distal portion 202 includes a plurality of exterior flow regions 208a-c which have a perimeter, i.e. a circumference in the illustrated embodiment, that is smaller than that of adjacent regions of the distal portion. A plurality of slots 210 are located between the flow regions 208a-c, as are a plurality of protrusions 212. The distal portion 202 also includes a plurality of apertures 214 that extend from the exterior of the distal portion to the central lumen 204. So configured, cerebrospinal fluid (CSF) will be free to flow along the exterior of the catheter distal portion 202 from one flow region 208a-c to another, as well as in and out of the apertures 214, when distal portion regions 202a and 202b are in contact with tissue. Such flow of CSF, which is the result of the movement of the spine and beating of the heart, dilutes medication within the lumen 204 and apertures 214 that may be in contact with the arachnoid mater for prolonged periods. Thus, the configuration of the distal portion 202 reduces the likelihood that granulomas, which may be due to the prolonged exposure of the arachnoid mater to high concentration drugs, will form.

In the illustrated embodiment, the apertures 214 are rectangular in shape and are located in some of the slots 210. More specifically, there are four slots 210 and two diametrically opposed apertures 214 located between the flow regions 208a and 208b as well as four slots and two diametrically opposed apertures between the flow regions 208b and 208c. The apertures 214 between the flow regions 208b and 208c are offset from apertures between flow regions 208a and 208b by ninety degrees. It should be noted here, however, that the shape, number and location of the apertures 214 may be varied as desired, as may the shape, number and location of the flow regions 208a-c and slots 210. By way of example, but not limitation, the apertures 214 may be circular in shape and/or may be located in the flow regions 208a-c instead of the slots 210. In other implementations, the flow regions and slots may be eliminated. Here, the catheter body will simply be an tubular body with apertures of any suitable number, size and shape in the distal region.

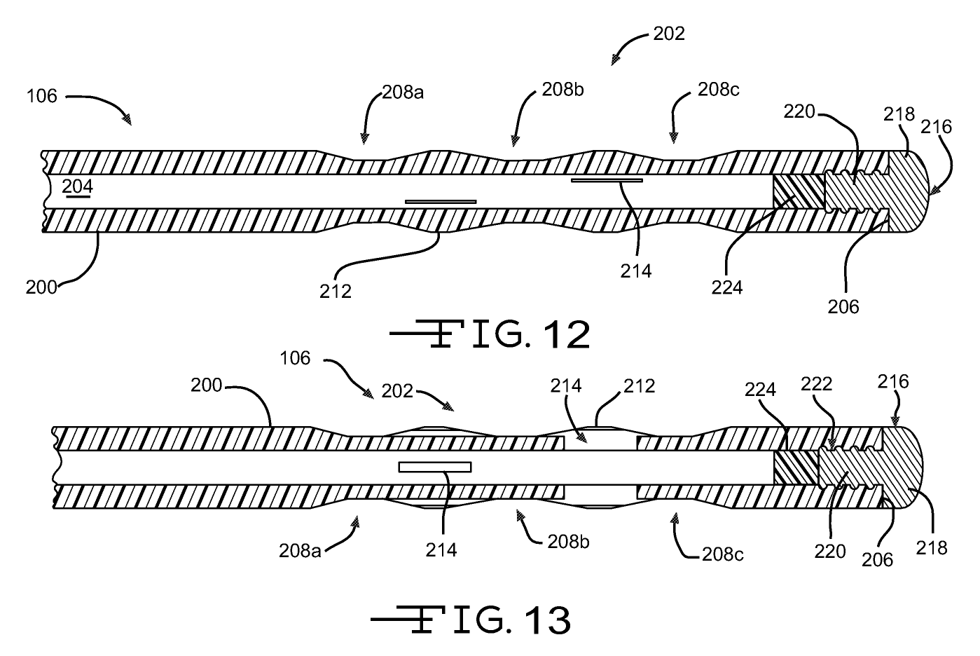

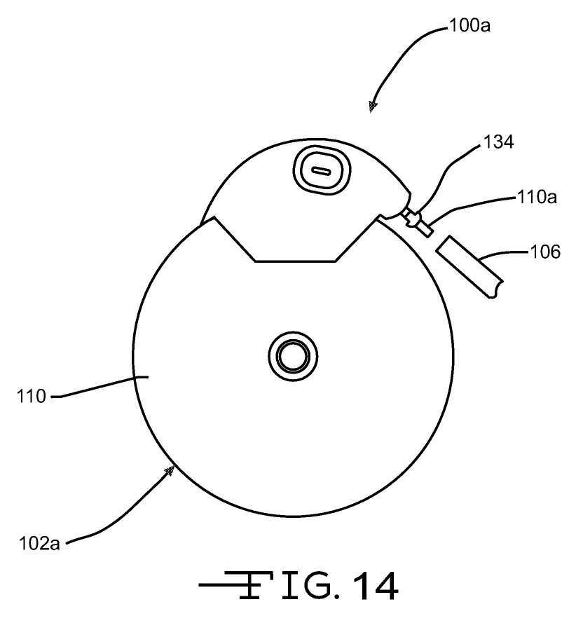

A marker tip 216 is carried on the distal end 206 of the catheter body 200. The exemplary marker tip 216 is radiopaque and, referring to FIGS. 12 and 13, includes a main portion 218 and a connector 220. The connector 220, which is located within the central lumen 204, has a plurality of indentations 222 such as, for example, the illustrated plurality of longitudinally spaced concentric grooves. The catheter distal portion 202 may be heated to its melting point after the marker tip connector 220 has been inserted into the central lumen 204 so that catheter material will flow into the indentations 222. In exemplary heating processes, hot air may be used to heat the catheter distal portion 114 and/or heat shrink tubing (e.g. polyimide or Teflon heat shrink tubing) may be positioned around the exterior of the catheter distal portion to control the catheter shape during the melting process. A mandrel (not shown) may also be inserted into the central lumen 204 proximal to the marker tip 216 prior to heating. The catheter material within the indentations 222, once cooled, secures the marker tip 216 to the catheter body 200. In other implementations, the connector may be smooth and secured to the catheter distal portion 202 with an adhesive. In still other implementations, marker tips may be configured such that they can be mounted on the catheter body distal end 206, and cover the distal end of the central lumen 204, without a connector that extends into the central lumen.

The exemplary subarachnoid catheter 106 illustrated in FIGS. 11-13 is also provided with an abutment 224 that is located within the central lumen 204 proximal to the marker tip 216. The exemplary abutment 224, which is cylindrical in shape and has an outer diameter (OD) that is equal to the inner diameter (ID) of the catheter body 200, may be formed from any suitable material and secured to the catheter distal portion 202 with an adhesive. The abutment 224 may, alternatively, be an integral portion of the catheter body 200. Abutments may also be formed by injecting a hardenable substance (e.g. room temperature vulcanizing silicone rubber adhesive) into the central lumen 204. The abutment 224 prevents stylets from separating the marker tip 216 from the distal end 206 of the catheter body 200 as the stylet is pushing the distal portion 202 of the catheter 106 to a target location within, for example, the subarachnoid space around the spinal cord.

With respect to materials, suitable materials for the connector 110 include metals (e.g. titanium) and hard plastics. Suitable materials for the catheter body 200 include, but are not limited to, polymers such as polyurethane (e.g. Carbothane® 95A), silicone, polyethylene, and polypropylene. Carbothane® 95A has higher tensile strength and tear resistance than, for example, silicone. As such, Carbothane® 95A facilitates the application of greater retention forces and the use of sharper barb edges than would be practicable with a weaker material such as silicone, thereby reducing the likelihood of a catheter disconnect as compared to weaker materials. Suitable materials for the marker tip 216 include, but are not limited to, radiopaque materials such as platinum, gold, tungsten, barium filled plastics, and iridium.

With respect to dimensions, the dimensions of the connector 110 will depend to some extent on the dimensions and material of the associated catheters. The exemplary catheter body 200, which is configured for use in the subarachnoid space, is circular in cross-section and has an OD of about 0.055 inches and an ID of about 0.021 inches. Here, the connector tube support 116 may have an OD of about 0.028 inches and the barb 134 may have an OD at the apex 138 of about 0.044 inches. The OD of the catheter body 200 at the exterior flow regions 208a-c is about 0.042 inches, and adjacent exterior flow regions are about 0.1 inch apart. The present catheters are not, however, limited to a circular cross-sectional shape. The length of the catheter body 200 may also vary from about 10 inches to about 40 inches, depending on the intended application.

It should be noted here that in other connector implementations, a second barb, such as one of the exemplary barbs 134-134e, may be associated with a connector. In the exemplary context of connector 110, a barb may be associated with the support tube 118. Other connectors in accordance with the present inventors may be more closely associated with an implantable pump or other infusion device. To that end, the exemplary infusion device 100a illustrated in FIG. 14 includes a pump 102a, a catheter 106 and a connector 110a with a barb 134. The pump 102a is identical to the pump 102 but for the connector 110a. Connectors in accordance with the present inventions may also form part of adapters that allow catheters to be connected to infusion devices where there is a catheter/outlet connector mismatch. One example of such an adapter includes a connector 110 and a short catheter (or other tube) that is configured to be connected to the pump outlet connector and is mounted on the support tube 118 of the connector 110. Connectors in accordance with the present inventions may also form part of a subcutaneous access port, such as a subcutaneous vascular access port, that includes a catheter connector.

It should also be noted here the although the connectors described above generally include a pair of indentations (and an associated pair of sharp edges), connectors in accordance with the present inventions may also include one, three, four or more indentations.

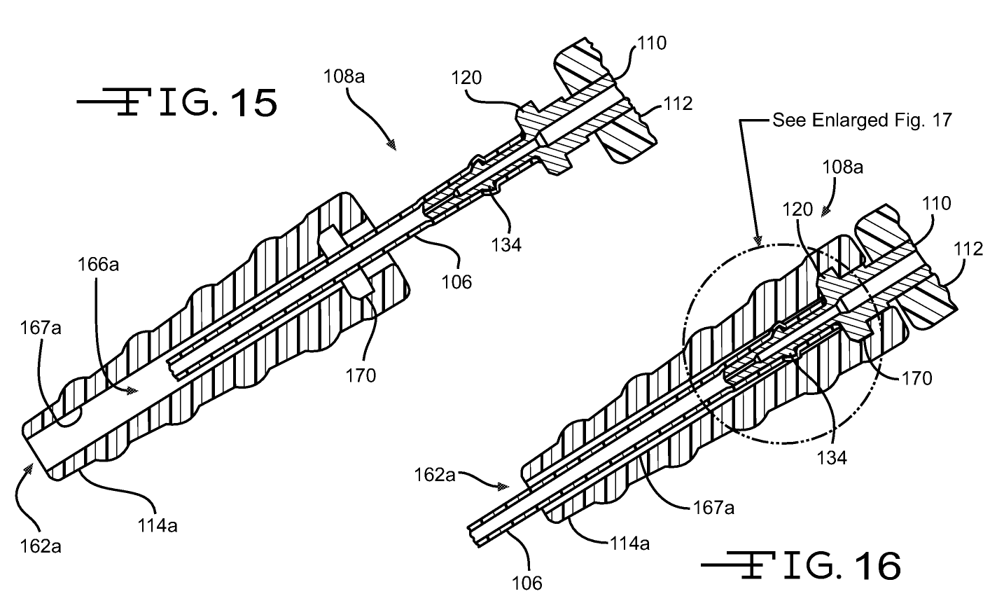

Other aspects of connector assemblies may be configured to reduce the likelihood of ESC cracking. For example, connector assemblies can be configured so as to prevent cells, which may adhere to the catheter, excrete acidic material and contribute to ESC cracking, from being aligned with the sharp edge of the barb where ESC cracking is most likely to occur. One example of such a connector assembly is generally represented by reference numeral 108a in FIGS. 15-17. Connector assembly 108a is substantially similar to connector assembly 108 and similar elements are represented by similar reference numerals. Additionally, although the exemplary connector assembly 108a includes barb 134, other barbs may be employed. Such barbs include, but are not limited to, the barbs 134a-134e illustrated in FIGS. 9A-9I.

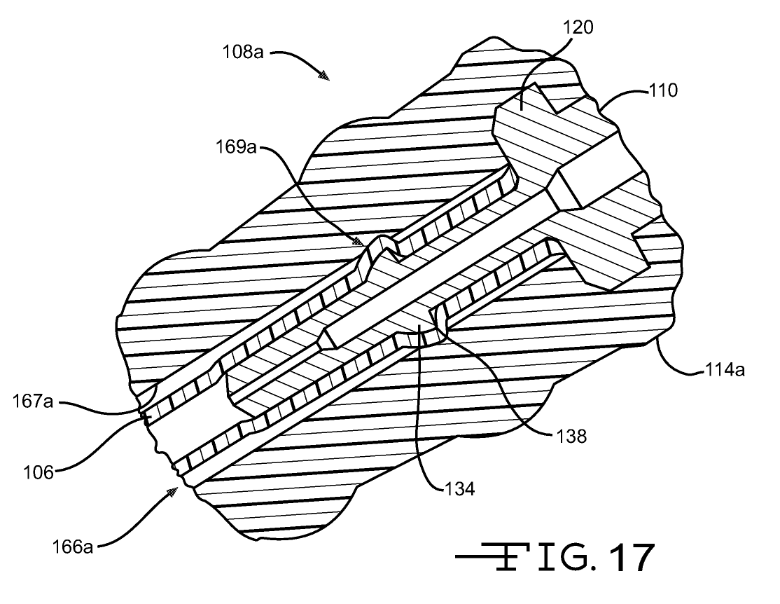

The exemplary connector assembly 108a illustrated in FIGS. 15-17 includes a connector 110 (with a barb 134), a first strain relief element 112 and a second strain relief element 114a. The second strain relief element 114a has an internal lumen 162a with a relatively narrow region 166a for the catheter 106 and a relatively wide region 170 for the connector flange 120. The barb 134 and the relatively narrow region 166a are respectively sized and shaped such that the inner surface 167a of the strain relief element 114a (which defines the internal lumen 162a) will engage, at a minimum, the outer surface of a portion of the catheter 106 that is aligned with the barb when the strain relief element is moved from the position illustrated in FIG. 15 to the position illustrated in FIGS. 16 and 17. In the illustrated implementation, the portion of the second strain relief element 114a that is aligned with the barb 134 will be compressed and, accordingly, will form a seal 169a (FIG. 17) with the outer surface of the associated portion of the catheter 106. The seal 169a will prevent cells from adhering to the portion of the catheter that is aligned with the sharp edges 148 and 150 (FIG. 7).

The diameter of the internal lumen 162a of the second strain relief element 114a in the illustrated embodiment is less than the diameter of the portion of the catheter that is aligned with barb apex 138 (FIG. 17). Put another way, the diameter of the internal lumen 162a is less than the sum of the diameter of the barb 134 at the apex 138 and two times the wall thickness of the catheter 106. For example, in those instances where the wall thickness of the catheter is about 0.010 inch to about 0.020 inch, the diameter of the internal lumen 162a will be less than the sum of the diameter of the barb 134 at the apex 138 plus about 0.020-0.040 inch. In one exemplary implementation where barb 134 has an OD at the apex 138 of about 0.044 inch, the diameter of the internal lumen 162a would be less than about 0.064 inch if the catheter wall thickness was about 0.010 inch and would be less than about 0.084 inch if the catheter wall thickness was about 0.020 inch. In other exemplary implementations, the diameter of the internal lumen 162a may be substantially equal to the outer diameter of the catheter 106 or the OD of the connector support tube 116. Such a configuration would create a seal between the outer surface of the catheter and the inner surface of the strain relief element which extends along the length of the support tube 116, including the portion with the barb 134.

Accordingly, one method of combining a catheter with a connector assembly, whether at the design stage or the use stage, would entail taking one or more of the wall thickness of the catheter, the OD of the catheter, the OD of the support tube 116 and the OD of the barb apex 138 into account. The diameter of the strain relief element internal lumen 162a and the diameter of the barb 134 at the apex 138 may be selected as a function of catheter wall thickness. Alternatively, or in addition, in those instances where a particular catheter 106 with a given wall thickness (and/or outer diameter) is to be combined with a particular connector 110 with a given support tube and apex diameter, the second strain relief element 114a may be selected based on the diameter of its internal lumen 162a in order to create the desired seal.

Although the inventions disclosed herein have been described in terms of the preferred embodiments above, numerous modifications and/or additions to the above-described preferred embodiments would be readily apparent to one skilled in the art. The present inventions also include assemblies which consist of a catheter in combination with the connectors and connector assemblies described above and claimed below. It is intended that the scope of the present inventions extend to all such modifications and/or additions and that the scope of the present inventions is limited solely by the claims set forth below.

I claim:

1. A catheter connector, comprising:
a support tube defining a longitudinal axis; and
a barb, associated with the support tube, that defines a perimeter in a plane perpendicular to the longitudinal axis and includes at least one sharp edge that extends partially around the perimeter and at least one region without a sharp edge that extends partially around the perimeter and is longitudinally aligned with the sharp edge.

2. A catheter connector as claimed in claim 1, wherein the barb defines an apex and the at least one sharp edge is at the apex.

3. A catheter connector as claimed in claim 2, wherein
the support tube defines a free end and an outer surface that extends from the free end to the barb;
the perimeter of the barb increases gradually from the outer surface to the apex; and
the perimeter of the barb decreases abruptly from the apex.

4. A catheter connector as claimed in claim 1, wherein
the at least one sharp edge comprises a pair of spaced sharp edges; and
the at least one region without a sharp edge comprises a pair of spaced regions without a sharp edge located between the sharp edges.

5. A catheter connector as claimed in claim 1, wherein the perimeter comprises a circular perimeter.

6. A catheter connector as claimed in claim 1, wherein
the support tube defines a free end;
a flange is associated with the support tube; and
the barb is located between the free end and the flange.

7. A connector for use with a catheter, the connector comprising:
a support tube defining a longitudinal axis; and
means, associated with the support tube, for creating different levels of stress with longitudinally aligned regions of the catheter when the catheter is positioned over the support tube.

8. A connector as claimed in claim 7, wherein
the support tube defines a free end;
a flange is associated with the support tube; and
the means for creating different levels of stress is located between the free end and the flange.

9. A catheter connector, comprising:
a support tube defining a longitudinal axis; and
a barb associated with the support tube and defining a substantially circular cross-section in a plurality of longitudinally spaced planes oriented perpendicular to the longitudinal axis and a non-circular cross-section a plurality of longitudinally spaced planes oriented perpendicular to the longitudinal axis.

10. A catheter connector as claimed in claim 9, wherein
the barb defines an apex; and
the plurality of circular cross-sections are separated from the plurality of non-circular cross sections by the apex.

11. A catheter connector as claimed in claim 9, wherein the non-circular cross-sections include curved portions and flat portions.

12. A catheter connector as claimed in claim 9, wherein
the support tube defines a free end;
a flange is associated with the support tube; and
the barb is located between the free end and the flange.

13. A catheter connector, comprising:
a support tube defining a longitudinal axis; and
a barb, associated with the support tube, defining an exterior surface shaped like a zone of a sphere with first and second circumferentially spaced indentations formed therein.

14. A catheter connector as claimed in claim 13, wherein the circumferentially spaced indentations diametrically oppose one another.

15. A catheter connector as claimed in claim 13, wherein the circumferentially spaced indentations each include a surface that is substantially perpendicular to the longitudinal axis and a surface that is substantially parallel to the longitudinal axis.

16. A catheter connector as claimed in claim 13, wherein
the barb defines an apex; and
a portion of each of the circumferentially spaced indentations is aligned with the apex.

17. A catheter connector as claimed in claim 13, wherein
the support tube defines a free end;
a flange is associated with the support tube; and
the barb is located between the free end and the flange.

18. A catheter connector as claimed in claim 13, wherein portions of the circumferentially spaced indentations define sharp edges with adjacent portions of the exterior.

19. A connector for use with a catheter, the connector comprising:
a support tube defining a longitudinal axis, a support tube perimeter and a free end;
means for stretching a first portion of the catheter into a first shape having a first perimeter that is greater than the tube support perimeter; and
means for stretching a second portion of the catheter, which is located between the free end and the first portion of the catheter, into a second shape, which is different than the first shape, having a second perimeter that is greater than the tube support perimeter and is greater than the first perimeter.

20. A connector as claimed in claim 19, wherein the first shape comprises a non-circular shape and the second shape comprises a substantially circular shape.

21. A connector as claimed in claim 19, further comprising:
a sharp edge located at the intersection of the means for stretching a first portion of the catheter and the means for stretching a second portion of the catheter;
wherein the first and second portions of the catheter are equidistant from the sharp edge.

22. A connector as claimed in claim 19, further comprising:
a flange associated with the support tube such that the means for stretching a first portion of the catheter is located between the means for stretching a second portion of the catheter and the flange.

23. A catheter connector, comprising:
a support tube defining a longitudinal axis; and
a barb associated with the support tube and including first and second barb members, the first barb member including an outer surface without sharp edges and defining an apex, and the second barb member defining a perimeter in a plane perpendicular to the longitudinal axis and including at least one sharp edge that is longitudinally spaced from the apex of the first barb member and extends at least partially around the perimeter.

24. A catheter connector as claimed in claim 23, wherein the at least one sharp edge extends completely around the perimeter.

25. A catheter connector as claimed in claim 23, wherein the first barb member is longitudinally spaced from the second barb member.

26. A catheter connector as claimed in claim 23, wherein the first barb member is substantially spherical in shape.
